(19) United States
(12) Patent Application Publication
(10) Pub. No.: US 2012/0309003 A1
(43) Pub. Date: Dec. 6, 2012

(54) ENTEROHEMORRHAGIC E. COLI O104:H4 ASSAYS

(75) Inventors: CRAIG CUMMINGS, PACIFICA, CA (US); PIUS BRZOSKA, WOODSIDE, CA (US); ALAIN RICO, CHATENAY MALABRY (FR); YONGMEI ("ALEXIS") JI, CHICAGO, IL (US)

(73) Assignee: LIFE TECHNOLOGIES CORPORATION, CARLSBAD, CA (US)

(21) Appl. No.: 13/486,594

(22) Filed: Jun. 1, 2012

Related U.S. Application Data
(60) Provisional application No. 61/492,777, filed on Jun. 2, 2011.
(60) Provisional application No. 61/495,341, filed on Jun. 9, 2011.

(51) Int. Cl. C12Q 1/68; C07H 21/04

(52) U.S. Cl. 435/6.11; 536/23.7; 536/24.33; 536/24.32; 435/6.12

(57) ABSTRACT

Disclosed are compositions, methods and kits for the specific detection of enterohemorrahagic E. coli O104:H4 from contaminated samples including clinical samples, biological samples, food samples, complex food matrices, water, beverage samples, fermentation broth samples, forensic samples, environmental samples (e.g., soil, dirt, garbage, sewage, air, or water) as well as food processing and manufacturing surfaces.

### CROSS-REFERENCE TO RELATED APPLICATIONS

This application claims the benefit under 35 U.S.C. §119(e) of United States Provisional patent application U.S. Ser. No. 61/492,777 filed Jun. 2, 2011, and of United States Provisional patent application U.S. Ser. No. 61/495,341 filed Jun. 9, 2011, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy, created on May 24, 2012, is named LT00528U.txt and is 14,451,716 bytes in size.

### FIELD

The present teachings relate to compositions, methods and kits for specific detection and differentiation of pathogenic organisms. More particularly, the specification describes compositions and kits comprising nucleic acid sequences specific and/or unique to enterohemorrhagic E. coli O104:H4 and methods of use thereof. Methods for differentially detecting strains of enterohemorrhagic E. coli O104:H4 from other pathogens (including closely related serotypes and strains) are also described.

### BACKGROUND

Identification of bacterial contamination in food often occurs subsequent to an outbreak of a food borne illness. The bacterium Escherichia coli is frequently identified as a food contaminant of many food borne illnesses.

The recent deadly outbreak of E. coli related illnesses and deaths in Europe, in 2011, has been shown to be caused by a new hybrid type of pathogenic E. coli strain that is enterohemorrhagic. This new strain of bacterium is causative of Hemolytic Uremic Syndrome (HUS) which includes kidney failure and bloody diarrhea.

Another serotype known as E. coli O157:H7 is most often associated with outbreaks of food borne illness in the United States and elsewhere in the world and causes enterohemorrhagic colitis and possibly kidney failure. Detection of pathogenic E. coli, particularly serotypes causative of hemorrhagic colitis has become a public health priority. Regulations by the United States government require meat and other food processors to screen for the presence of strains such as O157:H7 in their finished products and more stringent guidelines are being considered in a number of states for the identification of O157:H7 in other commodities and food stuffs.

Many strains of genetically similar E. coli exist that vary dramatically in their pathogenicity. Genomic comparisons are revealing the consequences of genetic changes often underlie the emergence of new pathogenic bacteria. For example, E. coli O157:H7 has been determined to have evolved stepwise from the O55:H7 which is associated with infantile diarrhea. These two serotypes are more closely related at the nucleotide level while divergence was markedly different at the gene level. Likewise, other pathogenic serotypes have been shown to be less divergent at the nucleotide level making identification of pathogenic strains difficult.

Assays for the rapid, sensitive and specific detection of infectious pathogens are extremely important from both a public health and economic perspective. When new pathogenic strains arise there is a need for novel assays and methods for detecting and differentiating new pathogenic organisms from other pathogenic and non-pathogenic organisms to make a differential diagnosis of what is causing a particular disease. Furthermore, there is a need to develop methods to detect the presence of pathogenic organisms in food and other sources of contamination, especially when a new pathogenic strain is detected.

### SUMMARY

The present disclosure, in some embodiments, describes the identification of an enterohemorrhagic strain of E. coli from the recent European outbreaks of 2011 which is named E. coli O104:H4 also referred to as LB226692. In some embodiments, the present disclosure describes nucleic acid sequences corresponding to assembled genomes of E. coli O104:H4. SEQ ID. NOs: 108-SEQ ID NO: 471 comprise nucleic acid sequences corresponding to a genome assembly of E. coli O104:H4 (LB226692).

The present disclosure also describes nucleic acid sequences isolated from another enterohemorrhagic strain of E. coli from an older European outbreak of enterohemorrhagic disease in 2001, which is called HUSEC41, and is found to be phenotypically E. coli O104:H4 Stx2+eae⁻. The nucleic acid sequences of the genome assembly of HUSEC41 is described in SEQ ID NOs: 472-SEQ ID NO: 927.

In some embodiments, unique enterohemorrhagic E. coli O104:H4 nucleic acid sequences, also referred to herein as target signature sequences are described and comprise isolated nucleic acid molecules comprising a nucleotide sequence of SEQ ID NOS: 1-5, fragments thereof, and/or complements thereof. In some embodiments, unique enterohemorrhagic E. coli O104:H4 sequences may comprise isolated nucleic acid molecules comprising a nucleotide sequence having at least a 90% sequence identity, at least 80% sequence identity and/or at least 70% sequence identity to the nucleotide sequences of SEQ ID NO:1, SEQ ID NO:2, SEQ ID NO:3, SEQ ID NO:4, SEQ ID NO: 5, fragments thereof and/or complements thereof.

In some embodiments, E. coli O104:H4 isolated nucleic acid sequences may comprise nucleic acid molecules comprising at least 40 nucleotide sequence of SEQ ID NOS: 1-5; at least 30 nucleotide sequence of SEQ ID NOS: 1-5; at least 25 nucleotide sequence of SEQ ID NOS: 1-5; at least 20 nucleotide sequence of SEQ ID NOS: 1-5; at least 16 nucleotide sequence of SEQ ID NOS: 1-5; at least 15 nucleotide sequence of SEQ ID NOS: 1-5; at least 10 nucleotide sequence of SEQ ID NOS: 1-5; any intermediate number of contiguous sequences from at least about 10 nucleotides of sequence to at least about 40 nucleotides of sequence of SEQ ID NOS: 1-5 and sequences having 90% identity to the foregoing sequences.

In some embodiments, the disclosure describes compositions of isolated nucleic acid sequences having SEQ ID NOs: 6-104 fragments thereof, at least 10 contiguous nucleotide sequences thereof, complements thereof and isolated nucleic acid sequence comprising at least 90% nucleic acid sequence identity to the sequences set forth above.

In some embodiments, isolated nucleic acid sequence compositions of the disclosure may further comprise one or more label, such as, but not limited to, a dye, a radioactive isotope, a chemiluminescent label, a fluorescent moiety, a bioluminescent label an enzyme, and combinations thereof. In some embodiments, isolated nucleic acid sequences may be modified.

The disclosure also describes recombinant constructs comprising nucleic acid sequences unique to E. coli O104:H4 or isolated nucleic acid sequences described in this specification.

The present disclosure, in some embodiments, describes assay utilizing molecular methods such as sequence specific amplification and detection that offer significant improvements in speed, sensitivity and specificity over traditional microbiological methods. Embodiments relate to design and development of molecular detection assays comprising identification of a target sequence that is present in the enterohemorrhagic strains E. coli O104:H4 to be detected and absent or divergent in organisms not to be detected.

In some embodiments, methods of detecting the presence of enterohemorrhagic E. coli serotypes and strains in a sample are disclosed. In some embodiments, methods of detecting the presence of EHEC enterohemorrhagic E. coli serotypes referred to as EHEC strains in a sample are disclosed. In some embodiments, methods of detecting the presence of EAEC E. coli serotypes referred to as EHEC strains, which are less virulent strains, in a sample are disclosed.

In some embodiments, methods of detecting the presence of enterohemorrhagic E. coli O104:H4 in a sample are disclosed.

The specification also discloses methods for detection of an enterohemorrhagic E. coli O104:H4 organism from a sample and methods to exclude the presence of an enterohemorrhagic E. coli O104:H4 organism in a sample, wherein the detection of at least one nucleic acid sequence that is unique to an E. coli O104:H4 is indicative of the presence of an E. coli O104:H4 and the absence of detection of any nucleic acid sequence unique to an E. coli O104:H4 is indicative of the absence of an E. coli O104:H4 in the sample. Accordingly, a method of the disclosure, in some embodiments, may comprise detecting, in a sample, at least one (or more) nucleic acid sequence(s) having at least 10 to at least 25 nucleic acids of SEQ ID NO:1, SEQ ID NO:2, SEQ ID NO:3, SEQ ID NO:4, SEQ ID NO:5, and/or complementary sequences thereof and/or a sequence having 90% homology thereto, wherein detection of at least one nucleic acid sequence indicates the presence of an E. coli O104:H4 organism in the sample. Methods of detection may also comprise identification steps and may further comprise steps of sample preparation. Such embodiments are described in detail in sections below.

One embodiment method of detecting E. coli O104:H4 in a sample may comprise: detecting the presence of SEQ ID NO: 1 and/or a complement thereof and/or a fragment thereof; wherein detection of SEQ ID NO: 1 or a complement thereof or a fragment thereof confirms the presence of E. coli O104:H4 in a sample.

One embodiment method of detecting E. coli O104:H4 in a sample may comprise: detecting the presence of SEQ ID NO: 2 or a complement thereof or a fragment thereof; wherein detection of SEQ ID NO: 2 or a complement thereof or a fragment thereof confirms the presence of E. coli O104:H4 in a sample.

One embodiment method of detecting E. coli O104:H4 in a sample may comprise: detecting the presence of SEQ ID NO: 3 or a complement thereof or a fragment thereof; wherein detection of SEQ ID NO: 3 or a complement thereof or a fragment thereof confirms the presence of E. coli O104:H4 in a sample.

One embodiment method of detecting E. coli O104:H4 in a sample may comprise: detecting the presence of SEQ ID NO: 4 or a complement thereof or a complement thereof or a fragment thereof; wherein detection of SEQ ID NO: 4 or a complement thereof or a fragment thereof confirms the presence of E. coli O104:H4 in a sample.

One embodiment method of detecting E. coli O104:H4 in a sample may comprise: detecting the presence of SEQ ID NO: 5 or a complement thereof or a fragment thereof; wherein detection of SEQ ID NO: 5 or a complement thereof or a fragment thereof confirms the presence of E. coli O104:H4 in a sample.

In one embodiment, a method of detecting E. coli O104:H4 in a sample may comprise: detecting the presence of at least two sequences from SEQ ID NO: 1, SEQ ID NO: 2, SEQ ID NO: 3, SEQ ID NO: 4, SEQ ID NO: 5, or any complements and/or fragments thereof and/or a sequence having 90% homology thereto; wherein detection of at least two of these sequences confirms the presence of E. coli O104:H4 in a sample. In some embodiments, two of the sequences listed above may be detected. In some embodiments, at least three of the above sequences may be detected. In some embodiments detection of all the sequences SEQ ID. NO: 1-5 may detect the presence of E. coli O104:H4 in a sample.

In one embodiment, a method of detecting E. coli O104:H4 in a sample may comprise: detecting the presence of at least two sequences from SEQ ID NO: 1, SEQ ID NO: 2, SEQ ID NO: 3, SEQ ID NO: 4 SEQ ID NO: 5, or any complements thereof or any fragment thereof or a sequence having 90% homology thereto; wherein detection of at least two of these sequences confirms the presence of E. coli O104:H4 in a sample and not the presence of other E. coli O104 strains.

In other embodiments, not detecting at least one of a nucleic acid sequence selected from nucleotides described by either SEQ ID NOs: 1-5, at least 20 contiguous fragments thereof, or complements thereof can be used to exclude the presence of E. coli O104:H4 in a sample.

Fragments of a nucleic acid sequence may be described without limitation in various embodiments as nucleic acid sequences having at least 10 contiguous nucleic acids, nucleic acid sequences having at least 20 contiguous nucleic acids, and/or nucleic acid sequences having at least 25 contiguous nucleic acids.

Detection of the presence of target nucleic acids in a sample may be performed by a variety of methods, such as but not limited to, a nucleic acid amplification reaction and detection of an amplification product as indicative of the presence of the target nucleic acid. An amplification reaction can be an end-point determination. An amplification reaction can be a quantitative amplification reaction. For example a quantitative amplification can be a real-time PCR assay, such as but not limited to a SYBR® Green Assay or a TaqMan® Assay. Amplification reactions may be performed by using primer sequences designed to hybridize to and amplify target nucleic acid sequences such as but not limited to SEQ ID NOs: 1-5 described herein.

Detection of the presence of target nucleic acids in a sample may in some embodiments be performed by hybridization using probes specific to target sequences comprised in SEQ ID NO: 1, SEQ ID NO: 2, SEQ ID NO: 3, SEQ ID NO: 4, SEQ ID NO: 5, fragments and/or complements thereof and/or to sequences having 90% homology thereto. In some embodiments, combinations of amplification (to amplify a target nucleic acid sequence and/or a complement and/or a fragment thereof) and hybridization to detect one or more amplified products may be used for detection of the presence of target nucleic acids in a sample.

In one embodiment, disclosed is an assay for the detection of E. coli O104:H4 in a sample comprising a) hybridizing a first pair of PCR primers comprising a forward primer and a reverse primer operable to hybridize to at least a first target polynucleotide sequence (which may be in a non-limiting example a first target nucleic acid having either one of SEQ ID NOs: 1-5 or a complement or a fragment thereof or a sequence having 90% homology thereto); b) optionally hybridizing a second pair of PCR primers comprising a forward primer and a reverse primer operable to hybridize to at least a second target polynucleotide sequence (which may be in a non-limiting example a target nucleic acid having either one of SEQ ID NOs: 1-5 or a complement or a fragment thereof or a sequence having 90% homology thereto and being different from the first target polynucleotide sequence); c) amplifying the at least first and optionally the at least second target polynucleotide sequences; and d) detecting the at least first and optionally the at least second amplified target polynucleotide sequence products; wherein the detection of the at least first amplified target polynucleotide sequence product and optionally the detection of the at least second amplified target polynucleotide sequence product is indicative of the presence of E. coli O104:H4 in the sample.

In one embodiment, disclosed is an assay for the detection of E. coli O104:H4 in a sample comprising a) hybridizing a first pair of PCR primers selected from primer sequences from a row in Table 1 comprising a forward primer (selected from SEQ ID NOs: 6-26 and/or complements thereof and/or primers having 90% sequence homology thereto) and a corresponding reverse primer (selected from the same row from SEQ ID NOs: 27-47 and/or complements thereof and/or primers having 90% sequence homology thereto), to at least a first target polynucleotide sequence (also described in the same row as the first pair of primers are selected from in Table 1, described by fragment_number in the first column of the row); b) hybridizing a second pair of PCR primers selected from primer sequences in another row in Table 1 comprising a second forward primer and a second reverse primer (as described above), to at least a second target polynucleotide sequence (also described in the same row as the second pair of primers of Table 1); c) amplifying said at least first and said at least second target polynucleotide sequences; and d) detecting said at least first and said at least second amplified target polynucleotide sequence products; wherein the detection of the at least first amplified target polynucleotide sequence product and the detection of the at least second amplified target polynucleotide sequence product is indicative of the presence of E. coli O104:H4 in the sample.

In further embodiments, a method may further comprise detection of the first and second amplified products by hybridization using one or more probes. Probes that may be used are described in Table 1, where different probes specific to a particular target sequence of E. coli O104:H4 are described in SEQ ID NOs: 48-68. Primer-probe combinations outlined in a row of Table 1 may be used to amplify and detect a corresponding target nucleic acid sequence.

In some embodiments, hybridization may comprise using at least a first probe to detect a first amplification product and a second probe to detect a second amplified product, the first probe further comprising a first label and said second probe further comprising a second label, wherein both labels are selected from a dye, a radioactive isotope, a chemiluminescent label, and an enzyme, the dye comprises a fluorescein dye, a rhodamine dye, or a cyanine dye. In some non-limiting exemplary embodiments a label may be a dye such as a fluorescein dye and a first probe may be labeled with a FAM™ dye and a second probe may be labeled with a VIC® dye.

In some embodiments, a method of detection may further comprise preparing a sample for PCR amplification (prior to hybridizing with primers for amplification), and may comprise for example, but not limited to (1) bacterial enrichment, (2) separation of bacterial cells from the sample, (3) cell lysis, and (4) total DNA extraction.

Samples may include without limitation, clinical samples, food/beverage samples, water samples, and environmental sample. Food sample may comprise raw produce, meats as well as a selectively enriched food matrix.

Methods may include assays such as polymerase chain reactions, wherein hybridizing and amplifying of said first pair of polynucleotide primers occurs in a first vessel and said hybridizing and amplifying of said second pair of polynucleotide primers occurs in a second vessel, or hybridizing and amplifying of said first pair of polynucleotide primers and said hybridizing and amplifying of said second pair of polynucleotide primers occurs in a single vessel.

In another embodiment, the disclosure describes methods for detection of E. coli O104:H4 in a sample comprising: a) hybridizing a first pair of PCR primers to a first target polynucleotide sequence within SEQ ID NO:1 or SEQ ID NO:2 or SEQ ID NO:3 or SEQ ID NO:4 or SEQ ID NO:5; b) hybridizing a second pair of PCR primers to a second target polynucleotide sequence within a sequence selected from SEQ ID NO:1 or SEQ ID NO:2 or SEQ ID NO:3 or SEQ ID NO:4 or SEQ ID NO:5 but which is different from the first target polynucleotide sequence; c) amplifying said at least first and said at least second target polynucleotide sequences; and d) detecting said at least first and said at least second amplified target polynucleotide sequence products; wherein the detection of the at least first amplified target polynucleotide sequence product and the detection of the at least second amplified target polynucleotide sequence product is indicative of the presence of E. coli O104:H4 in the sample.

A method as described above may further comprise providing a first probe and a second probe the probes, wherein the first and second probes are different from each other, the first probe operable to identify the first amplified target polynucleotide sequence and the second probe operable to identify the second amplified target nucleotide sequence, the first probe further comprises a first label and said second probe further comprises a second label, wherein both labels are selected from a dye, a radioactive isotope, a chemiluminescent label, and an enzyme, the dye comprises a fluorescein dye, a rhodamine dye, or a cyanine dye; and hybridizing the first and second probes to the PCR amplified fragments to detect the presence of the first amplified target polynucleotide sequence and the second amplified target polynucleotide sequence from the sample.

Some embodiments describe methods for detecting E. coli O104:H4, comprising assays to detect the presence of multiple target genes of E. coli O104:H4 thereby detecting the presence of E. coli O104:H4. In some embodiments an assay may comprise detecting the presence of a fliC_H4 gene which detects an E. coli having a H4 allele of a fliC gene and/or detecting the presence of a terD gene that detects the presence of an E. coli having a terD tellurite resistance gene and/or detecting the presence of an O104 antigen encoding gene. In some embodiments, an assay may further comprise detecting the presence of a stx2 allele of a shigatoxin or a verotoxin gene comprising detecting an E. coli strain having a stx2 shiga-toxin encoding locus. In some embodiments, a method of the disclosure may further comprise detecting the presence of a locus called AAF-I which encodes aggregative fimbriae.

Methods comprising assay designs (including primer pairs and probes) for detecting the presence of two or more genes described above to detect and identify the presence of E. coli O104:H4 are described in the Table 2 of this application, according to some embodiments as well as in Tables 3 and 4 of this application.

Table 2 described several forward and reverse primer pairs and corresponding probe sequences that may be used to identify fliC-H4 genes, ter D genes, and O104 antigen genes. Table 3 described several forward and reverse primer pairs and corresponding probe sequences that may be used to identify stx2/VT2 genes. Table 4 describes described several forward and reverse primer pairs and corresponding probe sequences that may be used to identify the AAF-I locus.

In some embodiments, a method of the disclosure may comprise identifying one or more of these gene loci which are specific to the European 2011 outbreak E. coli O104:H4 strains. A method may further comprise providing multiple probes, wherein each probe is operable to hybridize to and identify one amplified target polynucleotide sequence specific to a different gene locus to be detected and each probe further comprises a different label such as but not limited to a dye, a radioactive isotope, a chemiluminescent label, and an enzyme, the dye comprises a fluorescein dye, a rhodamine dye, or a cyanine dye, to facilitate simultaneous detection of multiple target genes in a sample.

In another embodiment, disclosed is a kit for the detection of E. coli O104:H4 in a sample comprising: a) at least one pair of PCR primers specific to a target nucleic acid specific to an E. coli O104:H4 organism, wherein the PCR primers are operable to hybridize to and amplify under suitable conditions a target nucleic acid specific to an E. coli O104:H4 organism to form amplified target nucleic acid products; b) optionally further comprising probes specific to hybridize to and detect an amplified target nucleic acid product specific to an E. coli O104:H4 organism; and c) one or more components selected from at least one enzyme, dNTPs, at least one buffer, at least one salt, at least one control nucleic acid sample and an instruction protocol.

In some embodiments, primers of a kit may be sequences selected from a row in Tables 1 comprising a forward primer and a corresponding reverse primer (SEQ ID NO: 6-47) or fragments or complements or sequences having 90% homology thereto that are operable to hybridize to and amplify at least a first target polynucleotide sequence (also described in the same row as the first pair of primers of Table 1). If multiple primers (and probes) are comprised in a kit, a first PCR primer (and probe) is selected from any row of Table 1 and a subsequent (second, third . . . ) pair of PCR primers selected from another row in Table 1. In some embodiments, probes may be selected from SEQ ID NOs: 48-68. Primers and probes may be labeled and/or modified. A first probe may further comprise a first label and a second (or subsequent) probe further comprise a second (or subsequent) label. Kits may comprise more than one sets of primers and/or probes shown in Table 1. Kits in some embodiments may comprise all the primer and probes shown in Table 1.

In another embodiment, disclosed is a kit for the detection of E. coli O104:H4 in a sample comprising a) at least one pair of PCR primers selected from sequences selected from a row in the Tables 2, 3 and/or 4 described as forward primers and reverse primers such as SEQ ID NOS:69-82, and/or SEQ ID NOS:105-107 and/or SEQ ID NOS: 90-99, respectively, fragments and/or complements and/or sequences having 90% homology thereto, that are specific to bind to, hybridize and amplify at least one target polynucleotide sequence to form an amplified target nucleic acid sequence; and b) a optionally at least one probe specific to hybridize to and detect an amplified target nucleic acid product described in step a); and c) one or more components selected from at least one enzyme, dNTPs, at least one buffer, at least one salt, at least one control nucleic acid sample and an instruction protocol. Kits may comprise more than one sets of primers and/or probes shown in Tables 2, 3 and 4. Kits in some embodiments may comprise all the primer and probes shown in Tables 2, 3 and 4.

Kits may without limitation contain other buffers, other molecular bio reagents, and each component may be individually packaged or packaged in one or more container means.

In the following description, certain aspects and embodiments will become evident. It should be understood that a given embodiment need not have all aspects and features described herein. It should be understood that these aspects and embodiments are merely exemplary and explanatory and are not restrictive of the invention.

It is to be understood that both the foregoing general description and the following detailed description are exemplary and explanatory only and are not restrictive of the invention, as claimed.

### DETAILED DESCRIPTION

The present disclosure, in some embodiments, describes the identification of an enterohemorrhagic strain of E. coli from the 2011 European outbreaks which is an E. coli O104:H4 also referred to as LB226692. In some embodiments, the present disclosure describes nucleic acid sequences corresponding to assembled genomes of E. coli O104:H4. SEQ ID. NOs: 108-SEQ ID NO: 471 comprise nucleic acid sequences corresponding to a genome assembly of E. coli O104:H4 (LB226692).

Data obtained from DNA sequencing of this new bacterial strain, E. coli O104:H4 or LB226692, using the Ion Personal Genome Machine (Ion PGM™, Life Technologies Corporation) shows the presence of genes typically found in two different types of E. coli including enteroaggregative E. coli (EAEC) and enterohemorrhagic E. coli (EHEC). These results and further data analysis on the Ion PGM™, provide insight into this bacterium's aggressiveness and may help prevent further outbreaks.

The present disclosure also describes nucleic acid sequences isolated from another enterohemorrhagic strain of E. coli from an older European outbreak of enterohemorrhagic disease in 2001, which is called HUSEC41, and is found to be phenotypically E. coli O104:H4 Stx2+eae⁻. The nucleic acid sequences of the genome assembly of HUSEC41 is described in SEQ ID NOs: 472-SEQ ID NO: 927.

In some embodiments, the present disclosure describes isolated E. coli O104:H4 nucleic acid sequences comprising SEQ ID. NOs: 108-SEQ ID NO: 471, complements thereof, fragments thereof and nucleotides having 90% homology thereto, 80% homology thereto and 70% homology thereto.

In some embodiments, the present disclosure describes isolated E. coli O104:H4 nucleic acid sequences comprising SEQ ID. NOs: 472-SEQ ID NO: 927, complements thereof, fragments thereof and nucleotides having 90% homology thereto, 80% homology thereto and 70% homology thereto.

The present disclosure has, in some embodiments, identified strain and serotype unique DNA sequences of E. coli O104:H4 which have been utilized to design probes and primers and to design several methods for detection of various E. coli O104:H4 organisms, including for example, specific methods to detect specifically the 2011 European outbreak strain LB226692 as well as methods to specifically detect the HUSEC41 strain, using molecular biology techniques such as but not limited to PCR and hybridization.

The sequence of E. coli O104:H4 genome is described in SEQ ID NOs: 108-471. A bioinformatic approach to identify E. coli O104:H4 unique sequence regions was undertaken and evaluated by Sanger sequencing and real-time PCR assays.

In one embodiment of the current teachings bioinformatic and direct DNA sequencing comparisons were conducted in an effort to identify E. coli O104:H4 serotype-specific sequences. Alignment of the sequenced regions using algorithms identified at least five O104:H4 “unique” regions shown for example in SEQ ID NOs: 1-5. PCR primer pairs were designed for each of the unique regions to specifically amplify the unique sequences against both inclusion (organism to be detected, i.e., E. coli O104:H4) and exclusion genomes (organisms not to be detected, other E. coli non-O104:H4 serotypes).

In some embodiments, compositions of isolated nucleic acids may in some embodiments be describes as E. coli O104:H4 specific target nucleic acid sequences/regions. Some examples of E. coli O104:H4 specific target nucleic acid sequences/regions comprise isolated nucleic acid sequences having SEQ ID NOs: 1-5; including isolated nucleic acids comprising at least 40 nucleotide sequence of SEQ ID NOS: 1-5; at least 30 nucleotide sequence of SEQ ID NOS: 1-5; at least 25 nucleotide sequence of SEQ ID NOS: 1-5; at least 20 nucleotide sequence of SEQ ID NOS: 1-5; at least 16 nucleotide sequence of SEQ ID NOS: 1-5; at least 15 nucleotide sequence of SEQ ID NOS: 1-5; at least 10 nucleotide sequence of SEQ ID NOS: 1-5; any intermediate number of contiguous sequences from at least about 10 nucleotides of sequence to at least about 40 nucleotides of sequence of SEQ ID NOS: 1-5; sequences having 90% identity to the foregoing sequences; and complementary sequences thereto.

PCR primer pairs and probes for use in detection methods (such as but not limited to real-time PCR assays) were designed to these E. coli O104:H4 specific target nucleic acid regions. In some embodiments, the disclosure describes primer and probes designed herein as compositions of isolated nucleic acid sequences having SEQ ID NOs: 6-104 fragments thereof, at least 10 contiguous nucleotide sequences thereof, complements thereof and isolated nucleic acid sequence comprising at least 90% nucleic acid sequence identity to the sequences set forth above.

In some embodiments, the disclosure describes primer and probes designed herein as compositions of isolated nucleic acid sequences having SEQ ID NOs: 6-107 fragments thereof, at least 10 contiguous nucleotide sequences thereof, complements thereof and isolated nucleic acid sequence comprising at least 90% nucleic acid sequence identity to the sequences set forth above.

In some embodiments, isolated nucleic acid sequence compositions of the disclosure may further comprise one or more label, such as, but not limited to, a dye, a radioactive isotope, a chemiluminescent label, a fluorescent moiety, a bioluminescent label an enzyme, and combinations thereof. In some embodiments, isolated nucleic acid sequences may be modified.

The disclosure also describes recombinant constructs comprising nucleic acid sequences unique to E. coli O104:H4 or isolated nucleic acid sequences described in this specification including SEQ ID NOs: 1-927.

In some embodiments, primers and probes described above (and in Tables 1-4 below) can be used in one or more methods described herein (such as in one example real-time PCR assays on DNA extracts from various samples suspected of being contaminated) for identification of E. coli O104:H4. In all screening methods an internal positive control will have a detectable signal/positive result.

A method of the disclosure, in some embodiments, may comprise detecting, in a sample, at least one (or more) nucleic acid sequence(s) having at least 10 to at least 25 nucleic acids of SEQ ID NO:1, SEQ ID NO:2, SEQ ID NO:3, SEQ ID NO:4, SEQ ID NO:5, and/or complementary sequences thereof and/or a sequence having 90% homology thereto, wherein detection of at least one nucleic acid sequence indicates the presence of an E. coli O104:H4 organism in the sample. Methods of detection may also comprise identification steps and may further comprise steps of sample preparation.

One embodiment method of detecting E. coli O104:H4 in a sample may comprise: detecting the presence of SEQ ID NO: 1 and/or a complement thereof and/or a fragment thereof; wherein detection of SEQ ID NO: 1 or a complement thereof or a fragment thereof confirms the presence of E. coli O104:H4 in a sample.

One embodiment method of detecting E. coli O104:H4 in a sample may comprise: detecting the presence of SEQ ID NO: 2 or a complement thereof or a fragment thereof; wherein detection of SEQ ID NO: 2 or a complement thereof or a fragment thereof confirms the presence of E. coli O104:H4 in a sample.

One embodiment method of detecting E. coli O104:H4 in a sample may comprise: detecting the presence of SEQ ID NO: 3 or a complement thereof or a fragment thereof; wherein detection of SEQ ID NO: 3 or a complement thereof or a fragment thereof confirms the presence of E. coli O104:H4 in a sample.

One embodiment method of detecting E. coli O104:H4 in a sample may comprise: detecting the presence of SEQ ID NO: 4 or a complement thereof or a complement thereof or a fragment thereof; wherein detection of SEQ ID NO: 4 or a complement thereof or a fragment thereof confirms the presence of E. coli O104:H4 in a sample.

One embodiment method of detecting E. coli O104:H4 in a sample may comprise: detecting the presence of SEQ ID NO: 5 or a complement thereof or a fragment thereof; wherein detection of SEQ ID NO: 5 or a complement thereof or a fragment thereof confirms the presence of E. coli O104:H4 in a sample.

In one embodiment, a method of detecting E. coli O104:H4 in a sample may comprise: detecting the presence of at least two sequences from SEQ ID NO: 1, SEQ ID NO: 2, SEQ ID NO: 3, SEQ ID NO: 4, SEQ ID NO: 5, or any complements and/or fragments thereof and/or a sequence having 90% homology thereto; wherein detection of at least two of these sequences confirms the presence of E. coli O104:H4 in a sample. In some embodiments, two of the sequences listed above may be detected. In some embodiments, at least three of the above sequences may be detected. In some embodiments detection of all the sequences SEQ ID. NO: 1-5 may detect the presence of E. coli O104:H4 in a sample.

Detection of the presence of target nucleic acids in a sample may be performed by a variety of methods, such as but not limited to, a nucleic acid amplification reaction and detection of an amplification product as indicative of the presence of the target nucleic acid. An amplification reaction can be an end-point determination. An amplification reaction can be a quantitative amplification reaction. For example a quantitative amplification can be a real-time PCR assay, such as but not limited to a SYBR® Green Assay or a TaqMan® Assay. Amplification reactions may be performed by using primer sequences designed to hybridize to and amplify target nucleic acid sequences such as but not limited to SEQ ID NOs: 1-5 described herein.

Detection of the presence of target nucleic acids in a sample may in some embodiments be performed by hybridization using probes specific to target sequences comprised in SEQ ID NO: 1, SEQ ID NO: 2, SEQ ID NO: 3, SEQ ID NO: 4, SEQ ID NO: 5, fragments and/or complements thereof and/or to sequences having 90% homology thereto. In some embodiments, combinations of amplification (to amplify a target nucleic acid sequence and/or a complement and/or a fragment thereof) and hybridization to detect one or more amplified products may be used for detection of the presence of target nucleic acids in a sample.

The disclosed methods to identify E. coli O104:H4 results from a two-pronged approach to identify target sequences for E. coli O104:H4 that do not also detect closely related either pathogenic or non-pathogenic E. coli. In some embodiments, identified E. coli O104:H4 target sequences (for example, SEQ ID NOs: 1-5), were used to design primers and probes for real-time PCR assays. The subsequently designed PCR primers and probes for use in assays by real-time PCR detected unambiguously, specifically and with great sensitivity E. coli O104:H4. Some examples of primer and probes designed for this are described by SEQ ID NOs: 6-68 (see also Table 1 below).

In one embodiment, disclosed is an assay for the detection of E. coli O104:H4 in a sample comprising a) hybridizing a first pair of PCR primers comprising a forward primer and a reverse primer operable to hybridize to a first target polynucleotide sequence (which may be in a non-limiting example a first target nucleic acid having either one of SEQ ID NOs: 1-5 or a complement or a fragment thereof or a sequence having 90% homology thereto); b) amplifying the first target polynucleotide sequences; and d) detecting the first amplified target polynucleotide sequence products; wherein the detection of the first amplified target polynucleotide sequence product and optionally the detection of the at least second amplified target polynucleotide sequence product is indicative of the presence of E. coli O104:H4 in the sample. Accordingly, in one example detection of any one of the five unique target nucleic acid sequences SEQ ID NO: 1-5 may be used to detect the presence of an E. coli O104:H4 in the sample.

In some embodiments, neither assay alone may be definitive for a single serotype of E. coli due to genomic similarity between the genomic regions of other E. coli serotypes. Yet, when two assays, (for example, but not limited to the assays shown in Table 1), are used either in parallel or as a multiplex assay, (e.g., in a real-time TaqMan® assay), where each probe in each of the two assays has a different label for distinguishing results on a real-time PCR instrument, (e.g., a 7500 Fast Real-Time PCR System (Applied Biosystems)), a positive result from each assay is indicative of the presence of an enterohemorrahagic E. coli O104:H4.

For example, such a method may comprise: a) hybridizing a first pair of PCR primers comprising a forward primer and a reverse primer operable to hybridize to at least a first target polynucleotide sequence (which may be in a non-limiting example a first target nucleic acid having either one of SEQ ID NOs: 1-5 or a complement or a fragment thereof or a sequence having 90% homology thereto); b) hybridizing a second pair of PCR primers comprising a forward primer and a reverse primer operable to hybridize to at least a second target polynucleotide sequence (which may be in a non-limiting example a target nucleic acid having either one of SEQ ID NOs: 1-5 or a complement or a fragment thereof or a sequence having 90% homology thereto and being different from the first target polynucleotide sequence); c) amplifying the at least first and optionally the at least second target polynucleotide sequences; and d) detecting the at least first and optionally the at least second amplified target polynucleotide sequence products; wherein the detection of the at least first amplified target polynucleotide sequence product and optionally the detection of the at least second amplified target polynucleotide sequence product is indicative of the presence of E. coli O104:H4 in the sample. The method may also comprise hybridizing a third, a fourth and several different primers and repeating steps b) through d). Multiple amplifications and detections may be performed in parallel or simultaneously such as in a multiplex method.

In one example embodiment method for the detection of E. coli O104:H4 in a sample comprising a) hybridizing a first pair of PCR primers selected from primer sequences from a row in Table 1 comprising a forward primer (selected from SEQ ID NOs: 6-26 and/or complements thereof and/or primers having 90% sequence homology thereto) and a corresponding reverse primer (selected from the same row from SEQ ID NOs:27-47 and/or complements thereof and/or primers having 90% sequence homology thereto), to at least a first target polynucleotide sequence (also described in the same row as the first pair of primers are selected from in Table 1, described by fragment_number in the first column of the row); b) hybridizing a second pair of PCR primers selected from primer sequences in another row in Table 1 comprising a second forward primer and a second reverse primer (as described above), to at least a second target polynucleotide sequence (also described in the same row as the second pair of primers of Table 1); c) amplifying said at least first and said at least second target polynucleotide sequences; and d) detecting said at least first and said at least second amplified target polynucleotide sequence products; wherein the detection of the at least first amplified target polynucleotide sequence product and the detection of the at least second amplified target polynucleotide sequence product is indicative of the presence of E. coli O104:H4 in the sample. Optionally steps b)-d) above may be repeated with subsequent such as third, fourth . . . primer pairs.

In further embodiments, a method may further comprise detection of the first and second amplified (and subsequent such as third, fourth . . . ) products by hybridization using one or more probes. Probes that may be used are described in Table 1, where different probes specific to a particular target sequence of E. coli O104:H4 are described in SEQ ID NOs: 48-68. Primer-probe combinations outlined in a row of Table 1 may be used to amplify and detect a corresponding target nucleic acid sequence.

In other embodiments, dual or multiplex (more than 2 assay sets) assay approach can be used to detect and distinguish E. coli O104:H4 strains and serotypes from each other. Accordingly, some embodiments describe methods for specifically detecting E. coli O104:H4, comprising assays to detect the presence of multiple target genes of E. coli O104:H4 thereby detecting the presence of specific E. coli O104:H4 strains and serotypes.

In some embodiments, such a method may comprise detecting the presence of genes including a fliC_H4 gene which detects an E. coli carrying the H4 allele of the fliC gene in combination with detecting the presence of a terD gene that detects the presence of an carrying a terD tellurite resistance gene, the detection of an O104 antigen gene, an AAF-I gene and a Stx2 shiga-toxin encoding gene locus. Some embodiments describe detecting all these gene loci as positive identification of enterohemorrahagic E. coli O104:H4. Some embodiments describe detecting at least two of these gene loci as positive identification of enterohemorrahagic E. coli O104:H4. Some embodiments describe detecting two or more of these gene loci using specific primer combinations (and probes) described in Tables 2, 3 and 4 as positive identification of enterohemorrahagic E. coli O104:H4.

Some embodiments describe methods for detecting target gene sequences of enterohemorrahagic E. coli O104:H4 of the present European outbreak and distinguishing it from previous outbreak strains. Some example methods (assays) are described in Table 5.

Assay designs for detecting the presence of two or more genes to detect and identify the presence of E. coli O104:H4 are described in Tables 1-4 of this application and in Examples.

Table 2 described several examples of forward and reverse primer pairs and corresponding probe sequences that may be used to identify fliC-H4 and ter D, and O104 genes. Table 4 describes several examples of forward and reverse primer pairs and corresponding probe sequences that may be used to identify the AAF-I locus.

In some embodiments, a method of detection may further comprise preparing a sample for PCR amplification (prior to hybridizing with primers for amplification), and may comprise for example, but not limited to (1) bacterial enrichment, (2) separation of bacterial cells from the sample, (3) cell lysis, and (4) nucleic acid extraction (e.g. total DNA extraction).

In some embodiments, sample nucleic acid extraction may be bypassed and bacterial enrichment may be carried out by methods such as affinity separation, immunological separation and/or magnetic separation and PCR may be carried out on the enriched bacteria.

Compositions and methods described herein are ideally suited for the preparation of kits suitable for identifying the presence of E. coli O104:H4. Such a kit may comprise various reagents utilized in methods described above, preferably in concentrated form and/or in a lyophilized form. The reagents of this kit may comprise, but are not limited to, buffer, appropriate nucleotide triphosphates, DNA polymerases, intercalating dye, primers, probes, salt, and instructions for the use of the kit.

In one embodiment, disclosed is a kit for the detection of E. coli O104:H4 in a sample comprising: a) at least one pair of PCR primers specific to a target nucleic acid specific to an E. coli O104:H4 organism, wherein the PCR primers are operable to hybridize to and amplify under suitable conditions a target nucleic acid specific to an E. coli O104:H4 organism to form amplified target nucleic acid products; b) optionally further comprising probes specific to hybridize to and detect an amplified target nucleic acid product specific to an E. coli O104:H4 organism; and c) one or more components selected from at least one enzyme, dNTPs, at least one buffer, at least one salt, at least one control nucleic acid sample and an instruction protocol.

In some embodiments, kit primers may be labeled. A kit comprising multiple pairs of primers may have primer pairs each labeled with different labels that may be detectable separately. Probes comprised in kits of the disclosure may be labeled. If a kit comprises multiple probes each probe may be labeled with a different label to allow detection of different products that may be the target of each different probe.

In one example embodiment, primers of a kit may be sequences selected from a row in Tables 1 comprising a forward primer and a corresponding reverse primer (SEQ ID NO: 6-47) or fragments or complements or sequences having 90% homology thereto that are operable to hybridize to and amplify at least a first target polynucleotide sequence (also described in the same row as the first pair of primers of Table 1). If multiple primers (and probes) are comprised in a kit, a first PCR primer (and probe) is selected from any row of Table 1 and a subsequent (second, third . . . ) pair of PCR primers selected from another row in Table 1. In some embodiments, probes may be selected from SEQ ID NOs: 48-68. Primers and probes may be labeled and/or modified. A first probe may further comprise a first label and a second (or subsequent) probe further comprise a second (or subsequent) label. Kits may comprise more than one sets of primers and/or probes shown in Table 1. Kits in some embodiments may comprise all the primer and probes shown in Table 1.

In another example embodiment, disclosed is a kit for the detection of E. coli O104:H4 in a sample comprising a) at least one pair of PCR primers selected from sequences selected from a row in the Tables 2, 3 and/or 4 described as forward primers and reverse primers such as SEQ ID NOS:69-82, and/or SEQ ID NOS:105-107 and/or SEQ ID NOS: 90-99, respectively, fragments and/or complements and/or sequences having 90% homology thereto, that are specific to bind to, hybridize and amplify at least one target polynucleotide sequence to form an amplified target nucleic acid sequence; and b) a optionally at least one probe specific to hybridize to and detect an amplified target nucleic acid product described in step a); and c) one or more components selected from at least one enzyme, dNTPs, at least one buffer, at least one salt, at least one control nucleic acid sample and an instruction protocol. Kits may comprise more than one sets of primers and/or probes shown in Tables 2, 3 and 4. Kits in some embodiments may comprise all the primer and probes shown in Tables 2, 3 and 4.

Another example kit for the detection of E. coli O104:H4 may comprise: at least two PCR primer pairs operable to amplify at least two target genes of E. coli O104:H4, each PCR primers pair comprising at least one forward primer and at least one reverse primer; wherein the primers are selected from: at least one pair of primers operable to amplify a shiga-toxin encoding locus Stx2; and/or at least one primer pair operable to amplify an O104 antigen encoding locus; and/or at least one primer pair operable to amplify a fliC_H4 locus encoding a H4 allele of a fliC gene; and/or at least one primer pair operable to amplify a tellurite resistance gene terD; and/or at least one primer pair operable to amplify a locus encoding aggregative fimbriae AAF-I. The kit may additionally comprise at least two probes operable to bind to amplified products of at least two target genes of E. coli O104:H4 and in the example above may be selected from: at least one probe operable to hybridize to and detect an amplified product of a shiga-toxin encoding locus Stx2; and/or at least one probe operable to hybridize to and detect an amplified product of a O104 antigen encoding locus; and/or at least one probe operable to hybridize to and detect an amplified product of a fliC_H4 locus encoding a H4 allele of a fliC gene; and/or at least one probe operable to hybridize to and detect an amplified product of a tellurite resistance gene terD; and/or at least one probe operable to hybridize to and detect an amplified product of a locus encoding aggregative fimbriae AAF-I.

A kit of the disclosure may further comprise one or more components such as but not limited to: at least one enzyme, dNTPs, at least one buffer, at least one salt, at least one control nucleic acid sample, loading solution for preparation of the amplified material for electrophoresis, genomic DNA as a template control, a size marker to insure that materials migrate as anticipated in a separation medium, and an instruction protocol and manual to educate a user and limit error in use. It is within the scope of these teachings to provide test kits for use in manual applications or test kits for use with automated sample preparation, reaction set-up, detectors or analyzers. In some embodiments, a kit amplification product may be further analyzed by methods such as but not limited to electrophoresis, hybridization, mass spectrometry, nanostring, microfluidics, chemiluminescence and/or enzyme technologies.

Components of kits may be individually and in various combinations comprised in one or a plurality of suitable container means.

For the purposes of interpreting of this specification, the following definitions may apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “have” “having” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the phrase “nucleic acid,” “nucleic acid sequence” “oligonucleotide”, and polynucleotide(s)” are interchangeable and not intended to be limiting.

As used herein, the phrase “stringent hybridization conditions” refers to hybridization conditions which can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically “substantially complementary” to a second polynucleotide if hybridization occurs between the polynucleotide and the second polynucleotide. As used herein, “specific hybridization” or “hybridize” or “hybridization” refers to hybridization between two polynucleotides under stringent hybridization conditions.

As used herein, the term “polynucleotide” refers to a polymeric form of nucleotides of any length, either ribonucleotides, deoxynucleotides, or peptide nucleic acids (PNA), and includes both double- and single-stranded RNA, DNA, and PNA. A polynucleotide may include nucleotide sequences having different functions, including, for instance, coding regions, and non-coding regions such as regulatory regions. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. An “oligonucleotide” refers to a polynucleotide of the present invention, typically a primer and/or a probe.

As used herein a “target-specific polynucleotide” or a “target nucleic acid” refers to a polynucleotide having a target-binding segment that is perfectly or substantially complementary to a target sequence, such that the polynucleotide binds specifically to an intended target without significant binding to non-target sequences under sufficiently stringent hybridization conditions. The target-specific polynucleotide can be e.g., a primer or probe and the subject of hybridization with its complementary target sequence.

The term “target sequence”, “target signature sequence” “target nucleic acid”, “target” or “target polynucleotide sequence” refers to a nucleic acid of interest. The target sequence can be a polynucleotide sequence that is the subject of hybridization with a complementary polynucleotide, e.g. a primer or probe. The target sequence can be composed of DNA, RNA, an analog thereof, and including combinations thereof. The target sequence may be known or not known, in terms of its actual sequence and its amplification can be desired. The target sequence may or may not be of biological significance. As non-limiting examples, target sequences may include regions of genomic DNA, regions of genomic DNA which are believed to contain one or more polymorphic sites, DNA encoding or believed to encode genes or portions of genes of known or unknown function, DNA encoding or believed to encode proteins or portions of proteins of known or unknown function, DNA encoding or believed to encode regulatory regions such as promoter sequences, splicing signals, polyadenylation signals, etc.

As used herein an “amplified target polynucleotide sequence product” or “amplified product” refers to the resulting amplicon from an amplification reaction such as a polymerase chain reaction. The resulting amplicon product arises from hybridization of complementary primers to a target polynucleotide sequence under suitable hybridization conditions and the repeating in a cyclic manner the polymerase chain reaction as catalyzed by DNA polymerase for DNA amplification or RNA polymerase for RNA amplification.

As used herein, the “polymerase chain reaction” or PCR is a an amplification of nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., Eds, The Polymerase Chain Reaction, BirkHauser, Boston, Mass. (1994). Each of the above steps may be conducted at a different temperature, preferably using an automated thermocycler (Applied Biosystems LLC, a division of Life Technologies Corporation, Foster City, Calif.). If desired, RNA samples can be converted to DNA/RNA heteroduplexes or to duplex cDNA by methods known to one of skill in the art.

As used herein, “amplifying” and “amplification” refers to a broad range of techniques for increasing polynucleotide sequences, either linearly or exponentially. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods may comprise thermal-cycling or may be performed isothermally. In various embodiments, the term “amplification product” includes products from any number of cycles of amplification reactions.

In certain embodiments, amplification methods comprise at least one cycle of amplification, for example, but not limited to, the sequential procedures of: hybridizing primers to primer-specific portions of target sequence or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Molecular Cloning, Third Edition, Cold Spring Harbor Press (2000) (hereinafter “Sambrook and Russell”), Ausubel et al., Current Protocols in Molecular Biology (1993) including supplements through September 2005, John Wiley & Sons (hereinafter “Ausubel et al.”).

The term “label” refers to any moiety which can be attached to a molecule and: (i) provides a detectable signal; (ii) interacts with a second label to modify the detectable signal provided by the second label, e.g. FRET; (iii) stabilizes hybridization, i.e. duplex formation; or (iv) provides a capture moiety, i.e. affinity, antibody/antigen, ionic complexation. Labelling can be accomplished using any one of a large number of known techniques employing known labels, linkages, linking groups, reagents, reaction conditions, and analysis and purification methods. Labels include light-emitting compounds which generate a detectable signal by fluorescence, chemiluminescence, or bioluminescence (Kricka, L. in Nonisotopic DNA Probe Techniques (1992), Academic Press, San Diego, pp. 3-28). Another class of labels are hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2.sup.nd Edition, (1996) Oxford University Press, pp. 15-81). Yet another class of labels effect the separation or immobilization of a molecule by specific or non-specific capture, for example biotin, digoxigenin, and other haptens (Andrus, A. “Chemical methods for 5′ non-isotopic labelling of PCR probes and primers” (1995) in PCR 2: A Practical Approach, Oxford University Press, Oxford, pp. 39-54).

The terms “annealing” and “hybridization” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

The term “end-point analysis” refers to a method where data collection occurs only when a reaction is substantially complete.

The term “real-time analysis” refers to periodic monitoring during PCR. Certain systems such as the ABI 7700 Sequence Detection System (Applied Biosystems, Foster City, Calif.) conduct monitoring during each thermal cycle at a pre-determined or user-defined point. Real-time analysis of PCR with FRET probes measures fluorescent dye signal changes from cycle-to-cycle, preferably minus any internal control signals.

The term “quenching” refers to a decrease in fluorescence of a first moiety (reporter dye) caused by a second moiety (quencher) regardless of the mechanism.

A “primer,” as used herein, is an oligonucleotide that is complementary to a portion of target polynucleotide and, after hybridization to the target polynucleotide, may serve as a starting-point for an amplification reaction and the synthesis of an amplification product. Primers include, but are not limited to, spanning primers. A “primer pair” refers to two primers that can be used together for an amplification reaction. A “PCR primer” refers to a primer in a set of at least two primers that are capable of exponentially amplifying a target nucleic acid sequence in the polymerase chain reaction.

The term “probe” comprises a polynucleotide that comprises a specific portion designed to hybridize in a sequence-specific manner with a complementary region of a specific nucleic acid sequence, e.g., a target nucleic acid sequence. In certain embodiments, the specific portion of the probe may be specific for a particular sequence, or alternatively, may be degenerate, e.g., specific for a set of sequences. In certain embodiments, the probe is labeled. The probe can be an oligonucleotide that is complementary to at least a portion of an amplification product formed using two primers.

The term “modified” primer or probe may be used to describe a primer or probe modified as described for example in U.S. Pat. Nos. 7,408,051; 7,414,118; 7,585,649; 7,807,376; and U.S. patent application Ser. No. 12/889,273 (Attorney Docket No. 5099 Family); and U.S. Pat. Nos. 7,517,978; 7,943,752; and U.S. patent application Ser. No. 13/052,382 (Attorney Docket No. 5257 Family); and U.S. Pat. No. 7,408,051 and EP Patent No. 1902142B1, (Attorney Docket No. 5354 Family), the entire contents of which are incorporated herein by reference.

The terms “complement” and “complementary” as used herein, refer to the ability of two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary.

A “label” refers to a moiety attached (covalently or non-covalently), or capable of being attached, to an oligonucleotide, which provides or is capable of providing information about the oligonucleotide (e.g., descriptive or identifying information about the oligonucleotide) or another polynucleotide with which the labeled oligonucleotide interacts (e.g., hybridizes). Labels can be used to provide a detectable (and optionally quantifiable) signal. Labels can also be used to attach an oligonucleotide to a surface.

A “fluorophore” is a moiety that can emit light of a particular wavelength following absorbance of light of shorter wavelength. The wavelength of the light emitted by a particular fluorophore is characteristic of that fluorophore. Thus, a particular fluorophore can be detected by detecting light of an appropriate wavelength following excitation of the fluorophore with light of shorter wavelength.

The term “quencher” as used herein refers to a moiety that absorbs energy emitted from a fluorophore, or otherwise interferes with the ability of the fluorescent dye to emit light. A quencher can re-emit the energy absorbed from a fluorophore in a signal characteristic for that quencher, and thus a quencher can also act as a fluorophore (a fluorescent quencher). This phenomenon is generally known as fluorescent resonance energy transfer (FRET). Alternatively, a quencher can dissipate the energy absorbed from a fluorophore as heat (a non-fluorescent quencher).

As used herein the term “sample” refers to a starting material suspected of harboring a particular microorganism or group of microorganisms. A “contaminated sample” refers to a sample harboring a pathogenic microbe thereby comprising nucleic acid material from the pathogenic microbe. Examples of samples include, but are not limited to, food samples (including but not limited to samples from food intended for human or animal consumption such as processed foods, raw food material, produce (e.g., fruit and vegetables), legumes, meats (from livestock animals and/or game animals), fish, sea food, nuts, beverages, drinks, fermentation broths, and/or a selectively enriched food matrix comprising any of the above listed foods), water samples, environmental samples (e.g., soil samples, dirt samples, garbage samples, sewage samples, industrial effluent samples, air samples, or water samples from a variety of water bodies such as lakes, rivers, ponds etc.,), air samples (from the environment or from a room or a building), forensic samples, agricultural samples, pharmaceutical samples, biopharmaceutical samples, samples from food processing and manufacturing surfaces, and/or biological samples. A “biological sample” refers to a sample obtained from eukaryotic or prokaryotic sources. Examples of eukaryotic sources include mammals, such as a human, a cow, a pig, a chicken, a turkey, a livestock animal, a fish, a crab, a crustacean, a rabbit, a game animal, and/or a member of the family Muridae (a murine animal such as rat or mouse). A biological sample may include blood, urine, feces, or other materials from a human or a livestock animal. A biological sample can be, for instance, in the form of a single cell, in the form of a tissue, or in the form of a fluid.

A sample may be tested directly, or may be prepared or processed in some manner prior to testing. For example, a sample may be processed to enrich any contaminating microbe and may be further processed to separate and/or lyse microbial cells contained therein. Lysed microbial cells from a sample may be additionally processed or prepares to separate, isolate and/or extract genetic material from the microbe for analysis to detect and/or identify the contaminating microbe. Analysis of a sample may include one or more molecular methods. For example, according to some exemplary embodiments of the present disclosure, a sample may be subject to nucleic acid amplification (for example by PCR) using appropriate oligonucleotide primers that are specific to one or more microbe nucleic acid sequences that the sample is suspected of being contaminated with. Amplification products may then be further subject to testing with specific probes (or reporter probes) to allow detection of microbial nucleic acid sequences that have been amplified from the sample. In some embodiments, if a microbial nucleic acid sequence is amplified from a sample, further analysis may be performed on the amplification product to further identify, quantify and analyze the detected microbe (determine parameters such as but not limited to the microbial strain, pathogenicity, quantity etc.).

As used herein “preparing” or “preparing a sample” or “processing” or processing a sample” refers to one or more of the following steps to achieve extraction and separation of a nucleic acid from a sample: (1) bacterial enrichment, (2) separation of bacterial cells from the sample, (3) cell lysis, and (4) nucleic acid extraction and/or purification (e.g., DNA extraction, total DNA extraction, genomic DNA extraction, RNA extraction). Embodiments of the nucleic acid extracted include, but are not limited to, DNA, RNA, mRNA and miRNA. Alternatively, nucleic acid extraction and purification may be bypassed.

As used herein, “presence” refers to the existence (and therefore to the detection) of a reaction, a product of a method or a process (including but not limited to, an amplification product resulting from an amplification reaction), or to the “presence” and “detection” of an organism such as a pathogenic organism or a particular strain or species of an organism.

As used herein, “detecting” or “detection” refers to the disclosure or revelation of the presence or absence in a sample of a target polynucleotide sequence or amplified target polynucleotide sequence product. The detecting can be by end point, real-time, enzymatic, and by resolving the amplification product on a gel and determining whether the expected amplification product is present, or other methods known to one of skill in the art.

The presence or absence of an amplified product can be determined or its amount measured. Detecting an amplified product can be conducted by standard methods well known in the art and used routinely. The detecting may occur, for instance, after multiple amplification cycles have been run (typically referred to an end-point analysis), or during each amplification cycle (typically referred to as real-time). Detecting an amplification product after multiple amplification cycles have been run is easily accomplished by, for instance, resolving the amplification product on a gel and determining whether the expected amplification product is present. In order to facilitate real-time detection or quantification of the amplification products, one or more of the primers and/or probes used in the amplification reaction can be labeled, and various formats are available for generating a detectable signal that indicates an amplification product is present. For example, a convenient label is typically a label that is fluorescent, which may be used in various formats including, but are not limited to, the use of donor fluorophore labels, acceptor fluorophore labels, fluorophores, quenchers, and combinations thereof. Assays using these various formats may include the use of one or more primers that are labeled (for instance, scorpions primers, amplifluor primers), one or more probes that are labeled (for instance, adjacent probes, TaqMan® probes, light-up probes, molecular beacons), or a combination thereof. The skilled person will understand that in addition to these known formats, new types of formats are routinely disclosed. The present invention is not limited by the type of method or the types of probes and/or primers used to detect an amplified product. Using appropriate labels (for example, different fluorophores) it is possible to combine (multiplex) the results of several different primer pairs (and, optionally, probes if they are present) in a single reaction. As an alternative to detection using a labeled primer and/or probe, an amplification product can be detected using a polynucleotide binding dye such as a fluorescent DNA binding dye. Examples include, for instance, SYBR® Green dye or SYBR® Gold dye (Molecular Probes). Upon interaction with the double-stranded amplification product, such polynucleotide binding dyes emit a fluorescence signal after excitation with light at a suitable wavelength. A polynucleotide binding dye such as a polynucleotide intercalating dye also can be used.

The nucleic acid sequences described in SEQ ID NO: 108-471 are from a E. coli O104:H4 bacteria isolated from the European 2011 outbreak of an enterohemorrhagic strain of E. coli.

As used herein, an “E. coli O104:H4-specific nucleotide probe” refers to a sequence that is able to specifically hybridize to an E. coli O104:H4 target sequence present in a sample containing E. coli O104:H4 under suitable hybridization conditions and which does not hybridize with DNA from other E. coli strains or from other bacterial species. It is well within the ability of one skilled in the art to determine suitable hybridization conditions based on probe length, G+C content, and the degree of stringency required for a particular application.

It is expected that minor sequence variations in E. coli O104:H4-specific nucleotide sequences associated with nucleotide additions, deletions and mutations, whether naturally occurring or introduced in vitro, would not interfere with the usefulness of SEQ ID NO:1-5 and the various primer and probe nucleic acid sequences (SEQ ID NOS: 6-107) disclosed herein in the detection of enterohemorrhagic E. coli (EHEC), in methods for preventing EHEC infection, and in methods for treating EHEC infection, as would be understood by one of skill in the art. Therefore, the scope of the present invention as claimed is intended to encompass minor variations in the sequences of described here and sequences having at least 90% homology to the SEQ ID NO: 1-5 and the primer and probe sequences of SEQ ID NOS: 6-107 listed in Tables 1-4 in the Examples.

The probe may be RNA or DNA. Depending on the detection means employed, the probe may be unlabeled, radiolabeled, chemiluminescent labeled, enzyme labeled, or labeled with a dye. The probe may be hybridized with a sample in solution or immobilized on a solid support such as nitrocellulose, a microarray or a nylon membrane, or the probe may be immobilized on a solid support, such as a silicon chip or a microarray.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, may depend upon, for example, the nature of the nucleotide sequence, temperature, and buffer conditions. These conditions may also depend on what event is desired, such as hybridization, cleavage, or strand extension. An “isolated” polynucleotide refers to a polynucleotide that has been removed from its natural environment. A “purified” polynucleotide is one that is at least about 60% free, preferably at least about 75% free, and most preferably at least about 90% free from other components with which they are naturally associated.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims. Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.). The term “and/or” means one or all of the listed elements or a combination of any two or more of the listed elements.

There are many known methods of amplifying nucleic acid sequences including e.g., PCR. See, e.g., PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159 4,965,188 and 5,333,675 each of which is incorporated herein by reference in their entireties for all purposes.

Nucleic acid amplification techniques are traditionally classified according to the temperature requirements of the amplification process. Isothermal amplifications are conducted at a constant temperature, in contrast to amplifications that require cycling between high and low temperatures. Examples of isothermal amplification techniques are: Strand Displacement Amplification (SDA; Walker et al., 1992, Proc. Natl. Acad. Sci. USA 89:392 396; Walker et al., 1992, Nuc. Acids. Res. 20:1691 1696; and EP 0 497 272, all of which are incorporated herein by reference), self-sustained sequence replication (3SR; Guatelli et al., 1990, Proc. Natl. Acad. Sci. USA 87:1874 1878), the Qβ replicase system (Lizardi et al., 1988, BioTechnology 6:1197 1202), and the techniques disclosed in WO 90/10064 and WO 91/03573.

Examples of techniques that require temperature cycling are: polymerase chain reaction (PCR; Saiki et al., 1985, Science 230:1350 1354), ligase chain reaction (LCR; Wu et al., 1989, Genomics 4:560 569; Barringer et al., 1990, Gene 89:117 122; Barany, 1991, Proc. Natl. Acad. Sci. USA 88:189 193), transcription-based amplification (Kwoh et al., 1989, Proc. Natl. Acad. Sci. USA 86:1173 1177) and restriction amplification (U.S. Pat. No. 5,102,784).

Other exemplary techniques include Nucleic Acid Sequence-Based Amplification (“NASBA”; see U.S. Pat. No. 5,130,238), Q.beta. replicase system (see Lizardi et al., BioTechnology 6:1197 (1988)), and Rolling Circle Amplification (see Lizardi et al., Nat Genet 19:225 232 (1998)). The amplification primers of the present invention may be used to carry out, for example, but not limited to, PCR, SDA or tSDA. Any of the amplification techniques and methods disclosed herein can be used to practice the claimed invention as would be understood by one of ordinary skill in the art.

PCR is an extremely powerful technique for amplifying specific polynucleotide sequences, including genomic DNA, single-stranded cDNA, and mRNA among others. Various methods of conducting PCR amplification and primer design and construction for PCR amplification will be known to those of skill in the art. Generally, in PCR a double-stranded DNA to be amplified is denatured by heating the sample. New DNA synthesis is then primed by hybridizing primers to the target sequence in the presence of DNA polymerase and excess dNTPs. In subsequent cycles, the primers hybridize to the newly synthesized DNA to produce discreet products with the primer sequences at either end. The products accumulate exponentially with each successive round of amplification.

The DNA polymerase used in PCR is often a thermostable polymerase. This allows the enzyme to continue functioning after repeated cycles of heating necessary to denature the double-stranded DNA. Polymerases that are useful for PCR include, for example, Taq DNA polymerase, Tth DNA polymerase, Tfl DNA polymerase, Tma DNA polymerase, Tli DNA polymerase, and Pfu DNA polymerase. There are many commercially available modified forms of these enzymes including: AmpliTaq® and AmpliTaq Gold® both available from Applied Biosystems. Many are available with or without a 3- to 5′ proofreading exonuclease activity. See, for example, Vent® and Vent®. (exo-) available from New England Biolabs.

Other suitable amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554,517, and 6,063,603). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively.

Detection of E. coli O104:H4 by the use of the polymerase chain reaction provides a rapid method for detection. Moreover, the primer(s) (and probe used in a real-time PCR reaction) is desired to be specific and sensitive for only the organism of interest, i.e., E. coli O104:H4. However, the genome of E. coli O104:H4 is very similar to other E. coli genomes, both those which are disease causing (i.e., infectious) and pathogenic (ability to cause damage) e.g., serotype O104:H21 and others which may not be pathogenic. Therefore, in one embodiment, the identification and selection of genomic sequence from E. coli O104:H4 (e.g., SEQ ID NOs: 108-471, GenBank Accession No. ______) for the design of real-time PCR assays is based on the differential identification of E. coli O104:H4 genomic sequence (I, inclusion set) not found in closely related strains of E. coli (E, exclusion set). By identifying sequences not found in both infections and non-infectious strains of E. coli, target sequences specific to E. coli O104:H4 have been identified for primer design that do not, or only with rare exception, detect closely related E. coli strains and therefore identify E. coli O104:H4 with specificity and sensitivity.

The above description of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Those having ordinary skill in the art will understand that many modifications, alternatives, and equivalents are possible. All such modifications, alternatives, and equivalents are intended to be encompassed herein.

### EXAMPLES

The following procedures are representative examples of methods according to the disclosure that may be employed for the detection of E. coli O104:H4.

### Example 1

### Methods to Detect E Coli O104:H4 Using Target Signature Sequences

The present example describes assays designed to detect target signature sequences in E. coli O104:H4 using example probe and/or primer sequences designed by the present inventors. Primer sequences comprising pairs of forward and reverse primers and corresponding probe sequences are shown in Table 1 below.

An exemplary method of detecting the presence of E. coli O104:H4 in a sample, may comprise: isolating nucleic acid from a sample suspected of containing an E. coli O104:H4 and detecting the presence of at least one target signature sequences as shown in Table 1 below (shown in the first column). Each target signature sequence is shown in Table 1 by a fragment number and hit coordinate number corresponding to a nucleic acid sequence found in the genome of E. coli O104:H4 described in SEQ ID NOs: 108-471 and a BacTrack Assay Number in Table 1 describes an example of an associated primer pair (having a forward primer and a reverse primer) and a corresponding probe that may be used for amplification and detection of that target signature sequence. As shown in Table 1 a forward and reverse primer pair is shown in a row and if used in an amplification reaction will amplify the corresponding target signature sequence nucleic acid, for example BacTrack Assay number 61332 maybe used to detect the presence of E. coli O104:H4 in a sample suspected of being contaminated by detecting the presence of a target signature nucleic acid sequence corresponding to “fragment_{—}4942651-4942996” with hit coordinates “113 . . . 141 . . . 195” using a forward primer having the nucleic acid sequence ACTTCGAAATTTGCTGGCATGTG (SEQ ID NO: 6) and a reverse primer having the nucleic acid sequence TCATCTCCGCCAGGCATTTT (SEQ ID NO: 27) to amplify the target signature nucleic acid sequence, which may in some embodiments be detected using a probe having the nucleic acid sequence ATGTTGCCATTTGTTTGATTG (SEQ ID NO: 48).

TABLE 1

| Target Signature | Hit | BacTrack | | | |
|---|---|---|---|---|---|
| Sequence | coordinates | Assay ID | Forward Primer | Reverse Primer | Probe |
| fragment_4942651- | 113 . . . 141 . . . 195 | 61332 | ACTTCGAAATTTGCTGG | TCATCTCCGCCAGGC | ATGTTGCCATTTGTT |
| 4942996 | | | CATGTG | ATTTT | TGATTG |
| | | | [SEQ ID NO: 6] | [SEQ ID NO: 27] | [SEQ ID NO: 48] |
| fragment_4942651- | 218 . . . 243 . . . 282 | 61334 | ACAGAGCAAGCAAGACT | GAAATTGACTTCTGG | ATCCACAGGAATTTC |
| 4942996 | | | AGGAATG | CATGCTTCA | [SEQ ID NO: 49] |
| | | | [SEQ ID NO: 7] | [SEQ ID NO: 28] | |
| fragment_4942651- | 55 . . . 90 . . . 135 | 61340 | TTTCGCCAAACCAAAAT | CACATGCCAGCAAAT | AAGGCCTGCAAGAAC |
| 4942996 | | | CAGATAATGG | TTCGAAGTTA | T |
| | | | [SEQ ID NO: 8] | [SEQ ID NO: 29] | [SEQ ID NO: 50] |
| fragment_4942651- | 1 . . . 38 . . . 97 | 61342 | GTGAAAAATGAAACGTA | CAGGCCTTTAAGATC | TTCTTCTCTACAATA |
| 4942996 | | | TCGCTATTTTGT | GCCATTATC | GTTTTCG |
| | | | [SEQ ID NO: 9] | [SEQ ID NO: 30] | [SEQ ID NO: 51] |
| fragment_4942651- | 173 . . . 194 . . . 241 | 61347 | ACAAAAATGCCTGGCGG | CATTCCTAGTCTTGC | AAACGCTGTAAGAAC |
| 4942996 | | | AGAT | TTGCTCTGT | TC |
| | | | [SEQ ID NO: 10] | [SEQ ID NO: 31] | [SEQ ID NO: 52] |
| fragment_5101462- | 15 . . . 46 . . . 120 | 61330 | TCGAACAAGTTTCCTTT | TGCATGAGAATCATG | TAGTGTGTTTATTTT |
| 5102841 | | | AATGCAACAA | TAGTTACATTTTGGT | GAAAAAATAA |
| | | | [SEQ ID NO: 11] | [SEQ ID NO: 32] | [SEQ ID NO: 53] |
| fragment_5101462- | 48 . . . 93 . . . 140 | 61331 | ATTTTTTCAAAATAAAC | TGCTTTATCACTACA | CAAAATGTAACTACA |
| 5102841 | | | ACACTATTTAATAGCTT | CGTCTTGCAT | TGATTCTC |
| | | | GCTAAT | [SEQ ID NO: 33] | [SEQ ID NO: 54] |
| | | | [SEQ ID NO: 12] | | |
| fragment_5101462- | 207 . . . 245 . . . 295 | 61336 | ACACGATCAATAATCAC | GTTGCACAGTCGATA | TTAGCCATTTTCATT |
| 5102841 | | | TACACTCAAATCA | CCCTAATGTA | TTCC |
| | | | [SEQ ID NO: 13] | [SEQ ID NO: 34] | [SEQ ID NO: 55] |
| fragment_5101462- | 151 . . . 189 . . . 246 | 61341 | CCACACTAAAAACCTTC | CCTTCTGTGTTGATT | CCTTTACACACCTAA |
| 5102841 | | | GAATTTCGT | TGAGTGTAGTGA | TCAAC |
| | | | [SEQ ID NO: 14] | [SEQ ID NO: 35] | [SEQ ID NO: 56] |
| fragment_5101462- | 117 . . . 141 . . . 199 | 61345 | TGCAAGACGTGTAGTGA | GTGTGTAAAGGACTT | TTTAGTGTGGTGATT |
| 5102841 | | | TAAAGCA | TTTTCACTACGAA | TAGCC |
| | | | [SEQ ID NO: 15] | [SEQ ID NO: 36] | [SEQ ID NO: 57] |
| fragment_5131553- | 122 . . . 142 . . . 188 | 61333 | CGCGAAGCAGCAAAACT | GTTGCGCCAGCATAC | TCCCGCCAACTATACG |
| 5131745 | | | TGA | TTCTG | [SEQ ID NO: 58] |
| | | | [SEQ ID NO: 16] | [SEQ ID NO: 37] | |
| fragment_5131553- | 23 . . . 46 . . . 87 | 61338 | CCCGTCATGAGAGAACT | CGAGTTTCGTAGGCC | ACAGCTTTCCATAGTA |
| 5131745 | | | GAACT | AGATCAA | ATCA |
| | | | [SEQ ID NO: 17] | [SEQ ID NO: 38] | [SEQ ID NO: 59] |
| fragment_5131553- | 61 . . . 95 . . . 129 | 61348 | GCTGTTTGATCTGGCCT | GCTTCGCGGAGCAAT | ACCAACCGGACTCGCG |
| 5131745 | | | ACGAAA | GTG | [SEQ ID NO: 60] |
| | | | [SEQ ID NO: 18] | [SEQ ID NO: 39] | |
| fragment_5298507- | 58 . . . 94 . . . 152 | 61329 | GCGAGGGATGCGCATAA | ACCCTCACGAGTTAA | CAGTGGCTAAAGATAT |
| 5299488 | | | AA | GTTGTTTTGT | TC |
| | | | [SEQ ID NO: 19] | [SEQ ID NO: 40] | [SEQ ID NO: 61] |
| fragment_5298507- | 206 . . . 237 . . . 291 | 61335 | AGCCCAACATCTTTGAT | CGCCCAGGCATTAAA | CATCGCTCATTGACTT |
| 5299488 | | | TGTACTCA | TGAACCTA | TAA |
| | | | [SEQ ID NO: 20] | [SEQ ID NO: 41] | [SEQ ID NO: 62] |
| fragment_5298507- | 163 . . . 196 . . . 241 | 61339 | CTCGAGGCCAAATTTCT | CGATGCCCCTTTGAG | TTGGGCTGGAGTTAAC |
| 5299488 | | | TCTTTAAATAGG | TACAATCA | G |
| | | | [SEQ ID NO: 21] | [SEQ ID NO: 42] | [SEQ ID NO: 63] |
| fragment_5298507- | 112 . . . 141 . . . 207 | 61344 | ATTGCGAATAACCATCA | CTGGAGTTAACGAAG | ACTCGTGAGGGTAGAA |
| 5299488 | | | CAAAACAACT | GCCTATTTAAAGA | AT |
| | | | [SEQ ID NO: 22] | [SEQ ID NO: 43] | [SEQ ID NO: 64] |
| fragment_5298507- | 5 . . . 38 . . . 96 | 61346 | CGTTTATCACACTGATA | CTGGCATTTGCTGGT | ACCACAAAGGGAGTCT |
| 5299488 | | | TTCAGAATCACTACT | GGTTT | TTT |
| | | | [SEQ ID NO: 23] | [SEQ ID NO: 44] | [SEQ ID NO: 65] |
| fragment_5351200- | 185 . . . 245 . . . 299 | 61328 | AGGCCAAATGGCCATAT | TGCGGCTGCTTATTA | AAGAAATGACTTAGAA |
| 5352643 | | | TTTTAAAATAGTTATC | TGTTAACTGT | GATTATCTC |
| | | | [SEQ ID NO: 24] | [SEQ ID NO: 45] | [SEQ ID NO: 66] |
| fragment_5351200- | 57 . . . 94 . . . 137 | 61337 | CGCTAACGCGGCCATAT | GCGAAACCCTGCAGA | TCGCTGATTATCTTTC |
| 5352643 | | | TAATTAAA | TGGA | ATCATATT |
| | | | [SEQ ID NO: 25] | [SEQ ID NO: 46] | [SEQ ID NO: 67] |
| fragment_5351200- | 117 . . . 137 . . . 198 | 61343 | ACTCCATCTGCAGGGTT | TGGCCATTTGGCCTA | CCCACCAGTATAAATT |
| 5352643 | | | TCG | CAACATAT | GTG |
| | | | [SEQ ID NO: 26] | [SEQ ID NO: 47] | [SEQ ID NO: 68] |

Detecting the presence of at least one target signature sequences may also comprise methods such as amplification, hybridization, mass spectrometry, nanostring, microfluidics, chemiluminescence, enzyme technologies and combinations thereof.

Detecting the presence of an amplified product of a target signature sequence using probes as described above may comprise providing at least a first probe (and in some embodiments additional probes), the first probe operable to identify the first amplified target polynucleotide sequence, the first probe further comprises a first label (which may be a dye, a radioactive isotope, a chemiluminescent label, and an enzyme, the dye comprises a fluorescein dye, a rhodamine dye, or a cyanine dye); and hybridizing the first probes to a PCR amplified fragment to detect the presence of a first amplified target polynucleotide sequence from the sample, which is indicative of the presence of a target signature sequence which in turn is indicative of the presence of E. coli O104:H4 in a sample.

Some embodiments of a method may comprise detection of at least one target signature nucleic acid sequence to detect the presence of E. coli O104:H4 in a sample. Some embodiments of a method may comprise detecting the presence of at least two target signature nucleic acid sequences to detect the presence of E. coli O104:H4 in a sample. In some embodiments, detection of at least one of these nucleic acid sequences confirms the presence of E. coli O104:H4 in a sample while the absence of detection of one or more of the signature sequences rules out the presence of E. coli O104:H4 in the sample.

### Example 2

### Methods to Detect E. Coli O104:H4 by Detecting Combinations of Genes/Genetic Loci Found in an Outbreak Strain

In some embodiments methods are described comprising assays designed to detect combinations of multiple genes that are present in the 2011 European outbreak E. Coli O104:H4 strains. These specific combinations of genes have been identified as being present in the outbreak strains and are not present in other closely related strains and serotypes. Accordingly, assays of the disclosure comprise using primer pairs and probe sequences designed to selectively amplify these genes present in the outbreak strains of E. Coli. Detection of combinations of these genes indicates the presence of an outbreak strain.

According to some embodiments, detecting the presence combinations of two or more target genes of E. coli O104:H4 selected from a shiga-toxin encoding locus Stx2, an O104 antigen encoding locus, a fliC_H4 locus encoding a H4 allele of a fliC gene, and a terD tellurite resistance gene comprising using probes and primer sequences described in Table 2 below (having SEQ ID NOS: 69-89) for detecting an O104 antigen encoding locus, a fliC_H4 locus encoding a H4 allele of the fliC gene, and a terD tellurite resistance gene and the use of probes and primer sequences comprising SEQ ID NOS: 105-107 (Table 3) for detecting the Stx2 locus are described.

According to some embodiments, detecting the presence combinations of target genes of E. coli O104:H4 selected from a shiga-toxin encoding locus Stx2, an O104 antigen encoding locus, a fliC_H4 locus encoding a H4 allele of the fliC gene, a terD tellurite resistance gene and a locus called AAF-I, which encodes aggregative fimbriae, comprising using probes and primer sequences described in Table 2 below (having SEQ ID NOs: 69-89) for detecting an O104 antigen encoding locus, a fliC_H4 locus encoding a H4 allele of the fliC gene, and a terD tellurite resistance gene; and the use of probe and primer sequences comprising SEQ ID NOs: 105-107 (Table 3) for detecting an Stx2 locus are described; and the use of probes and primer sequences described in Table 4 below having nucleic acid sequences for detecting the AAF-1 locus are described comprising for example SEQ ID NOs: 90-104.

In some embodiments the detection of at least two of these target genes or gene encoding loci indicates the presence of the European outbreak strain E Coli O104:H4 (including strains LB226692 and TY-2482). In some embodiments, the detection of all these target genes or gene encoding loci indicates the presence of the European outbreak strain E Coli O104:H4 (including strains LB226692 and TY-2482). In some embodiments, the assays described are multiplex assays.

Table 2 below describes primer and probe sequences for detecting the presence of a fliC_H4 locus encoding a H4 allele of the fliC gene, a terD tellurite resistance gene and an O104 antigen encoding locus. The Assay ID # is used to indicate one example embodiment of an assay. For example, Assay ID #61324 in Table 2 describes an example method of detection comprising the use of forward primer GCAACAAATCGACAGTACCACTTT (SEQ ID NO: 69), reverse primer GTTTCGCCGCGCTGAA (SEQ ID NO: 76) and probe CGCCATGCCGGACAC (SEQ ID NO. 83), all present in the same row, for the detection of a fliC_H4 gene.

TABLE 2

| Assay ID | | | |
|---|---|---|---|
| and Gene | Forward Primer | Reverse Primer | Probe |
| fliC_H4 | | | |
| 61324 | GCAACAAATCGACAGTACCACTTT | GTTTCGCCGCGCTGAA | CGCCATGCCGGACAC |
| | [SEQ ID NO: 69] | [SEQ ID NO: 76] | [SEQ ID NO: 83] |
| 61325 | TGCGGATGTCAAGGATGCT | GCAGAATCAACGACCGCATATTTAG | CAGTGTAAGACAGTAAATTG |
| | [SEQ ID NO: 70] | [SEQ ID NO: 77] | [SEQ ID NO: 84] |
| 61326 | TGCGGATGTCAAGGATGCT | GCAGAATCAACGACCGCATATTTAG | ACACCGCGTCTAACAGT |
| | [SEQ ID NO: 71] | [SEQ ID NO: 78] | [SEQ ID NO: 85] |
| terD | | | |
| 61327 | GTAAAGCAGCTCCGTCAATGAAAA | CAAAGTCCTGACCGTCTGTTGA | ACGCGCATCCCAGCCAA |
| | [SEQ ID NO: 72] | [SEQ ID NO: 79] | [SEQ ID NO: 86] |
| O104 | | | |
| 61308 | CAGTTGGATAATCTGACAGGTGTCAT | GCGGAGGAAATTATTTCCGCAAT | ATACAGGTGTAATTCGTATTATC |
| | [SEQ ID NO: 73] | [SEQ ID NO: 80] | [SEQ ID NO: 87] |
| 61311 | TGCGCACCTGATCAAATTGTAATTG | TCGCACAATATCAATGGGTAAACGT | TAGCAAATCATCACTAATATAAC |
| | [SEQ ID NO: 74] | [SEQ ID NO: 81] | [SEQ ID NO: 88] |
| 61303 | TCCGTAATTGAAAAGCTTGGTGGAT | GCTGCAAGTATCCTAAGCCATAAGT | CATCATTATCTAATGGAAGATTAT |
| | [SEQ ID NO: 75] | [SEQ ID NO: 82] | [SEQ ID NO: 89] |

Assays described as Stx/VT2 (shiga toxin/verotoxin) may be used to detect an Stx locus and may include the use of the following probe and primer sequences as described in Table 3.

TABLE 3

| Gene ID # | Forward Primers | Reverse Primers | Probe |
|---|---|---|---|
| Stx/VT2 | GGGCAGTTATTTTGCTGTGGA | GAAAGTATTTGTTGCCGTATTAACGA | CGCGTTTTGACCATCT |
| | (SEQ ID NO: 105) | (SEQ ID NO: 106) | (SEQ ID NO: 107) |

Table 4 describes primer and probe sequences for detecting the presence of an AAF-1 locus. The Assay ID # is used to indicate one example embodiment assay. For example, Assay ID #61379 in this table comprises the use of forward primer GGCGGCTTGATTGTAGAGCAT (SEQ ID NO: 90), reverse primer CCAAGCGAATGTATTATCAGAGGATCA (SEQ ID NO: 95) and probe ACGGAGGTGTAATTATAAAAG (SEQ ID NO: 100), all present in the same row, for the detection of a AAF-1 locus.

TABLE 4

| Assay | | | |
|---|---|---|---|
| ID | Forward Primer | Reverse Primer | Probe |
| 61379 | GGCGGCTTGATTGTAGAGCAT | CCAAGCGAATGTATTATCAGAGGATCA | ACGGAGGTGTAATTATAAAAG |
| | [SEQ ID NO: 90] | [SEQ ID NO: 95] | [SEQ. ID NO: 100] |
| 61380 | GCAACTTGTGGAATTCCGAGTATT | CAGTAGCGATTGTGCTGACTTATCA | AAGCTACAGAAGACTATCATTTTA |
| | [SEQ ID NO: 91] | [SEQ ID NO: 96] | [SEQ ID NO: 101] |
| 61381 | ATCGCCTTTACACAGATCCATTGT | CGCCTTCGTATTGTCAAAACAGAA | ACGATCTATTGGAAAAGCTC |
| | [SEQ ID NO: 92] | [SEQ ID NO: 97] | [SEQ. ID NO.: 102] |
| 61382 | TGACTGCCCGGAAGACTTATTC | TTTGGGCCAGTTTGGAACCT | TCGGATCTTGGCGAATCA |
| | [SEQ ID NO: 93] | [SEQ ID NO: 98] | [SEQ ID NO: 103] |
| 61383 | ACTGCCCTCCTTTCTCAAGTAGA | TGTTTTCGAATAGAAAATGTAATCGCACAT | TTTGATGCCTCTTTATTAAAC |
| | [SEQ ID NO: 94] | [SEQ ID NO: 99] | [SEQ ID NO: 104] |

In the methods of the disclosure described, detecting each of the above referenced genes or gene loci to identify combination of genes, using the probes and primers described in this specification, may comprises using methods such as but not limited to amplification, hybridization, mass spectrometry, nanostring, microfluidics, chemiluminescence, enzyme technologies and combinations thereof. Amplification may comprise a polymerase chain reaction (PCR), RT-PCR, asynchronous PCR (A-PCR), and asymmetric PCR (AM-PCR), strand displacement amplification (SDA), multiple displacement amplification (MDA), nucleic acid strand-based amplification (NASBA), rolling circle amplification (RCA), transcription-mediated amplification (TMA). Methods may also further comprise isolating nucleic acid from a sample.

According to some embodiments, a method may comprise providing a first primer pair to amplify a first gene loci (selected from a shiga-toxin encoding locus Stx2, an O104 antigen encoding locus, a fliC_H4 locus encoding a H4 allele of the fliC gene, a terD tellurite resistance gene and a locus called AAF-I, which encodes aggregative fimbriae) and conditions for amplification to produce a first amplified target polynucleotide sequence. The method may then comprise detecting the first amplified target polynucleotide sequence (which corresponds to a first gene/locus as described above). In some embodiments, detecting may comprise providing a first probe operable to identify the first amplified target polynucleotide sequence. Since detecting a combination of genes described identifies an outbreak strain of E. coli O104:H4, a method generally may comprise detecting at least two of these genes. In some embodiments, a method may comprise detecting at least three of these genes/gene loci. In some embodiments, a method may comprise detecting at least four of these target geen/gene loci. In some embodiments, a method may comprise detecting detection of all five of these gene/gene loci.

Accordingly, a method of the disclosure, according to some embodiments, may comprise repeating the amplification and detection steps by providing at least a second primer pair to amplify a second gene/gene loci (and a third primer pair to amplify a third gene/gene loci; and a fourth primer pair to amplify a fourth gene/gene loci; and a fifth primer pair to amplify a fifth gene/gene loci) thereby amplifying a plurality of amplified target polynucleotide sequence (at least a second amplified target polynucleotide sequence (and in some embodiments at least a third, a fourth and a fifth amplified target polynucleotide sequence). Detection of the amplified target polynucleotide detects the presence of the corresponding gene or gene locus and presence of at least two (or more than two) of these genes/loci detects the presence of the outbreak strains of E. coli O104:H4.

In some embodiments, the present inventors have found that the assay designs described in Table 4 match and identify both outbreak strains of E. coli O104:H4 (LB226692 and TY-2482) but do not match and detect the historical HUSEC41 strain (the genomic sequence being comprised in SEQ ID NOs: 472-927).

### Example 3

### Methods to Distinguish Current 2011 European Outbreak Strains of E. Coli O104:H4 From Previous 2001 Outbreak Strain

Methods of the present disclosure, described above, were also screened against the HUSEC41 genome assembly described in SEQ ID NOs: 472-927, which is a historical European strain that caused hemolytic uremia syndrome (HUS) cases in the past. The HUSEC41 strain is phenotypically an E. coli O104:H4 Stx2+ eae−. The VT2 assays, designed to identify the Stx2 locus and fliC_H4 assays described in sections above perfectly matched this strain as well. HUSEC41 was also detected by the terD assay described above and several of the target signature nucleic acid sequence detecting assays (described in Example 1) also.

However, assays with ID numbers 61337 and 61343 (described in Table 1) were found to be completely specific for the two current E. coli O104:H4 outbreak isolates including the isolate described in the genome assemble described in SEQ ID NOs: 108-471 (also referred to as LB226692) as well as to the TY-2482 strain, and not to HUSEC41.

Accordingly, in some embodiments the present disclosure describes methods that are: 1) completely specific for the European 2011 outbreak strain (and may be used for e.g., for applications such as source tracking), as well as methods that are: 2) specific for the broader class of HUS-causing O104:H4 strains (e.g., for applications such as food safety testing).

Table 5 below describes various assay ID numbers (also described in previous tables and examples with details on probe and primer sequences) in relation to the specificity of strain detected.

TABLE 5

| | Strain of E. coli O104:H4 | | | |
|---|---|---|---|---|
| | LB226692 | | | |
| | (Sequences | | HUSEC41 | |
| | in SEQ | | (SEQ ID | |
| | ID NOs: | | NOs: | |
| Assay | 108-471) | TY2482 | 472-927) | |
| ID | (LTC) | (BGI) | (Muenster) | Specificity |
| 61342 | 0 | 0 | 0 | HUS-causing O104:H4 |
| 61340 | 0 | 0 | 0 | HUS-causing O104:H4 |
| 61332 | 0 | 0 | 0 | HUS-causing O104:H4 |
| 61347 | 0 | 0 | 0 | HUS-causing O104:H4 |
| 61334 | 0 | 0 | 0 | HUS-causing O104:H4 |
| 61336 | 0 | 0 | 0 | HUS-causing O104:H4 |
| 61341 | 0 | 0 | 0 | HUS-causing O104:H4 |
| 61345 | 0 | 0 | 0 | HUS-causing O104:H4 |
| 61331 | 0 | 0 | 0 | HUS-causing O104:H4 |
| 61330 | 0 | >250 | 0 | uncertain |
| 61346 | 0 | 0 | 0 | HUS-causing O104:H4 |
| 61329 | 0 | 0 | 0 | HUS-causing O104:H4 |
| 61344 | 0 | 0 | 0 | HUS-causing O104:H4 |
| 61339 | 0 | 0 | 0 | HUS-causing O104:H4 |
| 61335 | 0 | 0 | 0 | HUS-causing O104:H4 |
| 61337 | 0 | 0 | >250 | Outbreak-specific |
| 61343 | 0 | 0 | >250 | Outbreak-specific |
| 61328 | 0 | >250 | >250 | uncertain |
| 61338 | 0 | 0 | 158 | HUS-causing O104:H4* |
| 61333 | 0 | 0 | 20 | HUS-causing O104:H4* |
| 61348 | 0 | 0 | 60 | HUS-causing O104:H4* |

*detection of HUSEC41 may be weaker

Methods of the present disclosure were also screened against the two isolated strains of the current European outbreak, i.e., strain LB226692 which is described in the genome assemble described in SEQ ID NOs: 1 as well as to the TY-2482 strain. A summary of the assays performed and results are described below.

Both strains were detected by detection of the Stx2 using the VT2 assays; both strains were detected using the currently described fliC_H4 assays described in Assay ID Nos. 61324, 61325, 61326; both strains were detected using the currently described terD assay having Assay ID No. 61327; both strains were detected using the currently described O104 antigen assays having Assay ID No's. 61311, 61303, 61308; both strains were detected using the currently described target signature sequence assays of Table 1 with the exception of Assay ID No's 61328 and 61330.

While the foregoing specification teaches the principles of the present invention, with examples provided for the purpose of illustration, it will be appreciated by one skilled in the art from reading this disclosure that various changes in form and detail can be made without departing from the spirit and scope of the invention. These methods are not limited to any particular type of nucleic acid sample: plant, bacterial, animal (including human) total genome DNA, RNA, cDNA and the like may be analyzed using some or all of the methods disclosed in this disclosure. This disclosure provides powerful tools for analysis of complex nucleic acid samples. From experiment design to detection of E. coli O104:H4 assay results, the above disclosure provides for fast, efficient and inexpensive methods for detection of pathogenic E. coli O104:H4 strains.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. An isolated nucleic acid sequence having nucleic acid sequences comprising SEQ ID NO:1, SEQ ID NO:2, SEQ ID NO:3, SEQ ID NO:4, SEQ ID NO:5, fragments thereof, at least 25 contiguous nucleotide sequences thereof, complements thereof and sequences having at least 90% nucleic acid sequence identity thereto.

2. An isolated nucleic acid sequence having a nucleic acid sequence of any one of SEQ ID NOS:6-47, SEQ ID NOS:79-82, SEQ ID NOS: 90-88, fragments thereof, at least 25 contiguous nucleotide sequences thereof, complementary sequences thereof and labeled derivatives thereof, complements thereof and sequences having at least 90% nucleic acid sequence identity thereto.

3. An isolated nucleic acid sequence having a nucleic acid sequence of any one of SEQ ID NOS: 48-68, SEQ ID NOS:83-89, SEQ ID NOS: 100-104, complementary sequences thereof and labeled derivatives thereof and sequences having at least 90% nucleic acid sequence identity thereto.

4. A method of detecting the presence of E. coli O104:H4 in a sample, comprising:
detecting the presence of at least one target nucleic acid sequence specific to E. coli O104:H4 in the sample, wherein detection of at least one of target nucleic acid sequences confirms the presence of E. coli O104:H4 in the sample and the absence of a target nucleic acid sequence specific to E. coli O104:H4 is indicative of the absence of E. coli O104:H4 in the sample.

5. The method of claim 4 comprising detecting the presence of two or more target nucleic acid sequences specific to E. coli O104:H4 in the sample.

6. The method of claim 4, wherein the sample is a food sample, an agricultural sample, a produce sample, an animal sample, a clinical sample, an environmental sample, a biological sample, a water sample and an air sample.

7. The method of claim 4 comprising the steps of:
a) isolating nucleic acid from a sample to obtain sample nucleic acid;
b) hybridizing the sample nucleic acid with at least a first pair of polynucleotide primers specific to bind to a first target nucleic acid sequence specific to E. coli O104:H4;
c) amplifying the first target nucleic acid sequence specific to E. coli O104:H4 under suitable amplification conditions to obtain a first amplified target nucleic acid sequence product; and
d) detecting the first amplified target nucleic acid sequence product wherein the presence of the first amplified target nucleic acid product is indicative of the presence of E. coli O104:H4 in the sample and absence of the first amplified target nucleic acid product is indicative of the absence of E. coli O104:H4 in the sample.

8. The method of claim 7, further comprising repeating steps b) through d) using a second pair of polynucleotide primers specific to bind to a second target nucleic acid sequence to E. coli O104:H4; amplifying the second target nucleic acid sequence specific to E. coli O104:H4 under suitable amplification conditions to obtain the second amplified target nucleic acid sequence product; and detecting the second amplified target nucleic acid sequence product, wherein the presence of the first amplified target nucleic acid product and the second amplified target nucleic acid sequence product is indicative of the presence of E. coli O104:H4 in the sample and absence of the first amplified target nucleic acid product and the second amplified target nucleic acid product is indicative of the absence of E. coli O104:H4 in the sample.

9. The method of claim 4 wherein the target nucleic acid sequence specific to E. coli O104:H4 is SEQ ID NO:1, SEQ ID NO:2, SEQ ID NO:3, SEQ ID NO: 4 SEQ ID NO:5, a fragment thereof, at least a contiguous 25 nucleotide sequence thereof, a complement thereof, or a sequence having 90% identity thereto.

10. The method of claim 9, comprising detecting an enterohemorrahagic strain of E. coli O104:H4.

11. A method of distinguishing an E. coli O104:H4 from a non-O104:H4 E. coli strain comprising: detecting at least one of a nucleic acid sequence having SEQ ID NO:1, SEQ ID NO:2, SEQ ID NO:3, SEQ ID NO:4, SEQ ID NO:5, a fragment thereof, at least a contiguous 25 nucleotide sequence thereof, a complement thereof, or a sequence having 90% identity thereto, wherein detection of at least one of these nucleic acid sequences confirms the presence of an E. coli O104:H4 strain and the absence of detection confirms the presence of a non-O104:H4 E. coli strain.

12. A method of detecting the presence of E. coli O104:H4 in a sample, comprising:
detecting the presence of at least two target genes of E. coli O104:H4 selected from a shiga-toxin encoding locus Stx2, an O104 antigen encoding locus, a fliC_H4 locus encoding a H4 allele of the fliC gene, and a terD tellurite resistance gene comprising:
detecting the presence of at least one of these target genes by using probes and primer sequences comprising SEQ ID NOS: 69-89 and SEQ ID NOS: 105-107.

13. A method of detecting the presence of E. coli O104:H4 in a sample, comprising:
detecting the presence of at least two target genes of E. coli O104:H4 selected from a shiga-toxin encoding locus Stx2, an O104 antigen encoding locus, a fliC_H4 locus encoding a H4 allele of the fliC gene, a terD tellurite resistance gene and a AAF-I locus which encodes aggregative fimbriae, comprising:
detecting the presence of at least one of these target genes by using probes and primer sequences comprising SEQ ID NOS: 69-89, SEQ ID NOS:90-104 and SEQ ID NOS: 105-107.

14. The method of claim 17, comprising detecting an enterohemorrahagic strain of E. coli O104:H4.

15. A kit for the detection of E. coli O104:H4 comprising:
at least one pair of forward and reverse PCR primers operable to hybridize to and amplify at least one target nucleic acid sequence specific to E. coli O104:H4 under suitable amplification conditions to form an amplified target nucleic acid sequence product;
optionally at least one probe operable to hybridize to and detect the amplified target nucleic acid sequence product; and
one or more components selected from a group consisting of: at least one enzyme, dNTPs, at least one buffer, at least one salt, at least one control nucleic acid sample and an instruction protocol.

16. The kit of claim 15 wherein the primers comprise nucleic acid sequences of SEQ ID NOS: 6-47, SEQ ID NOS: 69-82, SEQ ID NOS: 90-99, SEQ ID NOS: 105-106, complements thereof, fragments thereof, sequences comprising at least 90% nucleic acid sequence identity thereto, or labeled derivatives thereof; and
the probe comprises SEQ ID NOS:48-68, SEQ ID NOS:83-89, SEQ ID NOS:100-104, SEQ ID NOS:107, complements thereof, fragments thereof, sequences comprising at least 90% nucleic acid sequence identity thereto, or labeled derivatives thereof.

17. A method for detecting an outbreak specific strain of E coli O104:H4 referred to as LB2266992 or strain TY-2482 and not detecting a HUSEC41 strain comprising:
a) isolating nucleic acid from a sample to obtain sample nucleic acid;
c) hybridizing the sample nucleic acid with at least a first pair of polynucleotide primers comprising a forward primer having SEQ ID NO: 25 and a reverse primer having SEQ ID NO: 46;
d) amplifying the first target nucleic acid sequence to form a first amplified target nucleic acid sequence product; and
e) detecting the at least first amplified target nucleic acid sequence product,
wherein detection of the at least first amplified target nucleic acid sequence product is indicative of the presence of E. coli O104:H4 strain referred to as LB2266992 or strain TY-2482 and is indicative of the absence of the HUSEC41 strain in the sample; and
wherein the detection may optionally comprise using a probe having SEQ ID NO: 67 to hybridize to and thereby detect the first amplified target nucleic acid sequence product.

18. A method for detecting an outbreak specific strain of E coli O104:H4 referred to as LB2266992 or strain TY-2482 and not detecting a HUSEC41 strain comprising:
a) isolating nucleic acid from a sample to obtain sample nucleic acid;
c) hybridizing the sample nucleic acid with at least a first pair of polynucleotide primers comprising a forward primer having SEQ ID NO: 26 and a reverse primer having SEQ ID NO: 47;
d) amplifying the first target nucleic acid sequence to form a first amplified target nucleic acid sequence product; and
e) detecting the at least first amplified target nucleic acid sequence product,
wherein detection of the at least first amplified target nucleic acid sequence product is indicative of the presence of E. coli O104:H4 referred to as LB2266992 or strain TY-2482 and is indicative of the absence of the HUSEC41 strain in the sample; and
optionally wherein the detection may comprise using a probe having SEQ ID NO: 68 to hybridize to and thereby detect the first amplified target nucleic acid sequence product.
